(19) United States
(12) Patent Application Publication
(10) Pub. No.: US 2008/0248575 A1
(43) Pub. Date: Oct. 9, 2008

(54) Drug and Gene Delivery by Polymer Nanonozzle and Nanotip Cell Patch

(75) Inventors: L. James Lee, Columbus, OH (US); Shengnian Wang, Columbus, OH (US); Yubing Xie, Cohoes, NY (US); Changchun Zeng, Tallahassee, FL (US); Chee Guan Koh, Lewis Center, OH (US); Zhengzheng Fei, Columbus, OH (US)

Correspondence Address: STANDLEY LAW GROUP LLP, 495 METRO PLACE SOUTH, SUITE 210, DUBLIN, OH, 43017, US

(73) Assignee: THE OHIO STATE UNIVERSITY RESEARCH FOUNDATION, Columbus, OH (US)

(21) Appl. No.: 12/090,958

(22) PCT Filed: Oct. 20, 2006
(86) PCT No.: PCT/US06/60105

§ 371 Date: May 27, 2008

Related U.S. Application Data
(60) Provisional application No. 60/728,465, filed on Oct. 20, 2005.

(51) Int. Cl. C12N 15/87; B29C 67/24; C12N 5/06

(52) U.S. Cl. 435/446; 264/225; 435/375

(57) ABSTRACT

Delivery of drugs or genes to individual cells is achieved on a nanoscale using electroporation techniques. In one method, a flow-through bioreactor having an inlet and an outlet connected by a flow chamber and a nanoporous membrane positioned in the flow chamber is used. Cells to be electroporated are flowed from the inlet to the outlet, a quantum of molecules of the at least one drug or gene in a fluid medium in the flow chamber. An electrical field applied in the flow chamber provides momentum to the molecules in the nanopores, resulting in delivery of the molecules into the plurality of cells.

### CROSS-REFERENCE TO RELATED APPLICATIONS

This application claims priority to U.S. provisional application Ser. No. 60/728,465, filed 20 Oct. 2005.

### TECHNICAL FIELD

The present invention relates to the use of polymer nanonozzles and nanotips to deliver drugs and/or genes to cells in an efficient and non-deleterious manner.

### BACKGROUND OF THE ART

Any efficient method for delivering a drug or a gene at the cell or tissue level is highly valuable for medical, pharmaceutical and gene engineering applications. Known techniques for delivering drug and genes into cells include intravascular injection of retrovirus and adenovirus, polyplex liposome particles, in vivo and ex vivo particle bombardment by gene gun, electroporation and combinations of these methods.

The actual choice of technique depends on the specific application and cell type. Viral transductions have high efficiency and could be engineered to deliver genes to many cell types, but risks of oncogenesis and inflammation are main concerns, especially after recent clinic accidents reported in both United States and Europe. Applications using non-viral methods are limited because if their low efficiency. Techniques such as the gene gun and electroporation are known to cause intolerably high physical damages to cells. Microinjection is widely used for gene transfer at the single cell level, but it is difficult to perform simultaneous injection on a large number of cells because of the lack of proper delivery devices.

As a result, there is still an unmet need in the art to provide a method and device for providing simultaneous injection of drugs or genes into a large number of cells in an efficient manner without causing damage to the cells.

### SUMMARY OF THE INVENTION

This and other previously unknown advantages are provided by the methods and devices enabled by the following detailed description.

### BRIEF DESCRIPTION OF THE DRAWINGS

A better understanding of the exemplary embodiments presented herein will be obtained when reference is made to the detailed description thereof and the accompanying drawing, in which identical parts are identified with identical reference numerals and wherein:

FIG. 1 is a diagrammatic representation of the steps in producing polymer nanonozzle and nanotip arrays;

FIG. 2 is a schematic representation of how the flow field in the centerline of a nanochannel varies under an electric field;

FIGS. 3a and 3b schematically illustrate two strategies for delivering drugs or genes into cells using a nanonozzle array;

FIGS. 4a through 4g show photomicrograph images demonstrating experimental results;

FIG. 5, schematically illustrates a drug or gene delivery technique in which the nanotip is retained in the cell treated (FIGS. 5a and 5b illustrate binding of the drug or gene onto the nanotip);

FIG. 6 schematically illustrates an embodiment of a batch-type membrane sandwich electroporation device;

FIGS. 7a though 7e depict results of bulk electroporation, localized electroporation and membrane sandwich electroporation experiments;

FIGS. 8a and 8b schematically illustrate two embodiments of hollow-fiber bioreactors for conducting membrane sandwich electroporation in a flow-through manner; and

FIGS. 9a through 9c schematically illustrate views of a hydrodynamic focusing bioreactor incorporating aspects of the membrane sandwich electroporation in a flow-through manner.

### DETAILED DESCRIPTION OF A PREFERRED EMBODIMENT

In the following detailed description, a method and a device are demonstrated for injecting drugs into a large number of cells simultaneously with arrays of nanonozzles or nanotips. In a nanonozzle, a conically shaped flow channel is capable of providing a great potential gradient when an electrical bias is applied. This results in an efficient way to either accelerate the rigid carriers (e.g., liposome particles, quantum dots or gold particles conjugated genes) to high momentum or to stretch flexible gene-containing biomolecules into a long worm-like shape with the radical size in several nanometers such that genes can be delivered into cells, by temporarily breaking through the cell membrane. Since the carriers and stretched genes have a radical size that is comparable to the natural pores on cell membrane, the damage to cells is minimized. In nanotips, a sacrificial template that is used to prepare a nanonozzle can itself be used for drug or gene delivery. The aperture of a nanotip can carry a specific dosage of the drug or gene. A short penetration of the cell by a tiny nanotip would not cause permanent damage to the cell, and the drug or gene is left inside the cell by dissolving in the surrounding medium when the remaining portion of the nanotip is pulled out from the cell membrane.

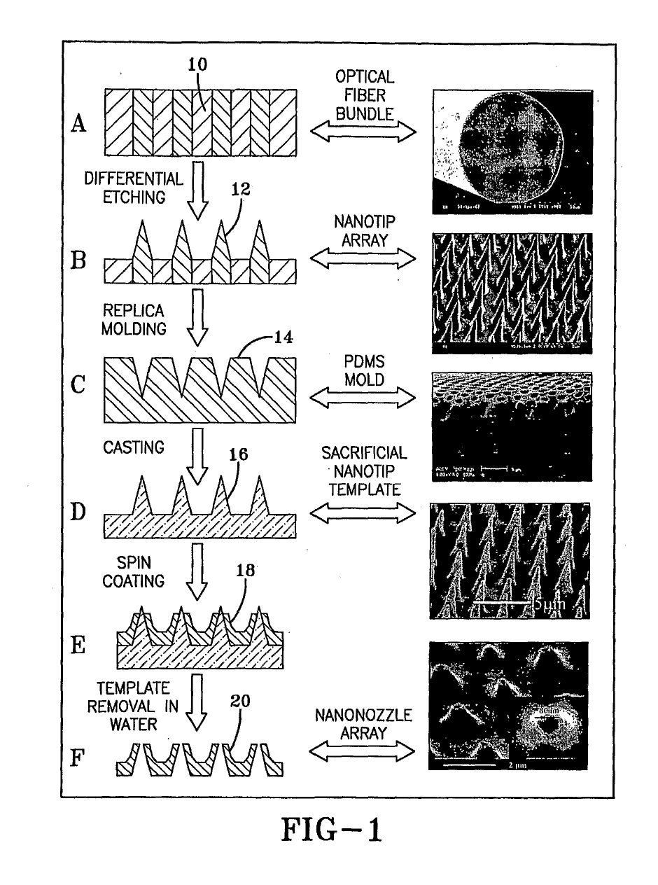

A novel low-cost process is used to produce a polymer nanonozzle and nanotip array. This process, referred to herein as “Sacrificial Template Imprinting” or STI, is diagrammatically represented in FIG. 1. In brief, a polymer template with conically shaped nanotips is fabricated first by a two-step replication using a female mold, typically comprising poly(dimethylsiloxane) (“PDMS”) female mold. These nanotips can directly be used as the carriers for drug/gene delivery or can be used as the sacrificial template in the fabrication of a nanonozzle array on a polymer layer. Because the polymer template can be removed by dissolving in water, removing the template will not result in structural damage or defects on the nanonozzle array. Removal of the template is a common difficulty encountered during de-molding in many nanofabrication processes. A typical density of such nanonozzle arrays is 10⁷ nozzles/cm² and could be as high as 10⁹ nozzles/cm². A typical nanonozzle is around 3 μm high with a channel diameter on the sharp end as small as 80 nm, and it can be made smaller. The converging ratio, that is, a ratio of the respective large end and small end diameters, can easily be as high as 30. In conjunction with silica synthesis on the channel surface, the nozzle size can be further reduced and the polymer structure can be reinforced.

Describing FIG. 1 in more detail, Step A involves providing an optical fiber bundle 10. In Step B, a technique, such as differential etching, is used to produce a nanotip array 12. In Step C, the nanotip array 12 is used to produce a replica mold 14, typically from PDMS. In Step D, the PDMS mold 14 can be used to produce a sacrificial nanotip template 16, using a technique such as casting. In Step E, the sacrificial nanotip template 16 has material built up on it, using a technique such as spin coating, resulting in a composite sacrificial template/nanonozzle array 18. Step F involves the removal, using water, of the sacrificial nanotip template 16 from the composite 18, resulting in the nanonozzle array 20.

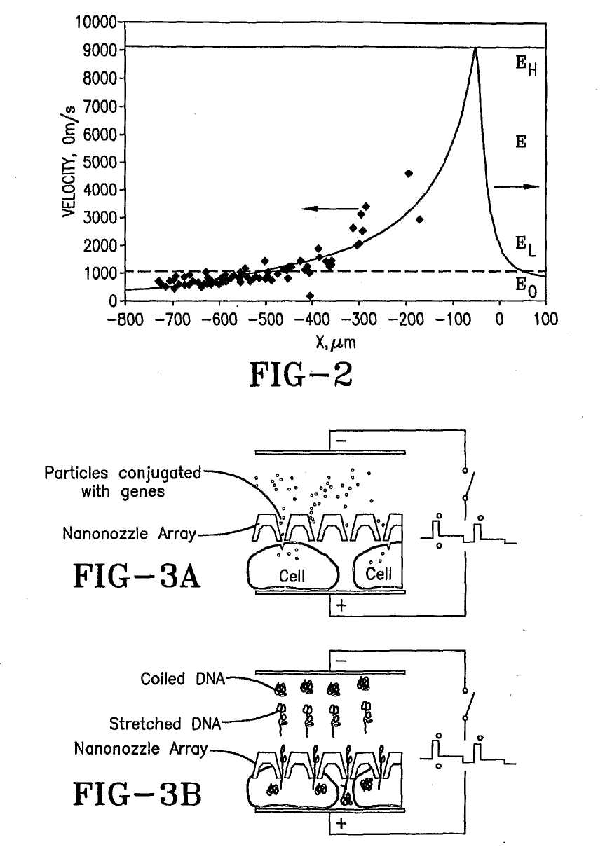

In the nanonozzle cell patch approach, a gentle electric bias is applied between two ends of a nanonozzle array, such as that constructed by the method of FIG. 1. The electric field strength (E) varies along the conical channel of the nanonozzles, due to the contraction of the cross section area. FIG. 2 shows the electric field strength based on calculation and the lateral velocity profile of polystyrene microspheres measured experimentally along the centerline of a 2D nozzle, which has the same geometry of nanonozzles used in this study but scaled up to 20 μm on the small end. The value of E jumps from the bulk value (Eo) to a slight higher value (E_{L}) at the inlet. Along the converging channels, the strength of the electric field increases parabolically to a high value at the outlet (E_{H}) and then descends drastically right outside the small end outlet to the bulk value (E_{O}). For example, for a nanonozzle with a converging ratio of 15, the magnitude of electric field near the small end (E_{H}) is around 225 times higher than that near the large end (E_{L}). It is obvious that the rapid increase in the electric field strength inside the converging nanonozzles is capable of accelerating the charged particles and molecules to a very high velocity at the outlet. Since this method relies on the electrophoretic (EP) movement of particles, the technique can be called an “EP Gun”.

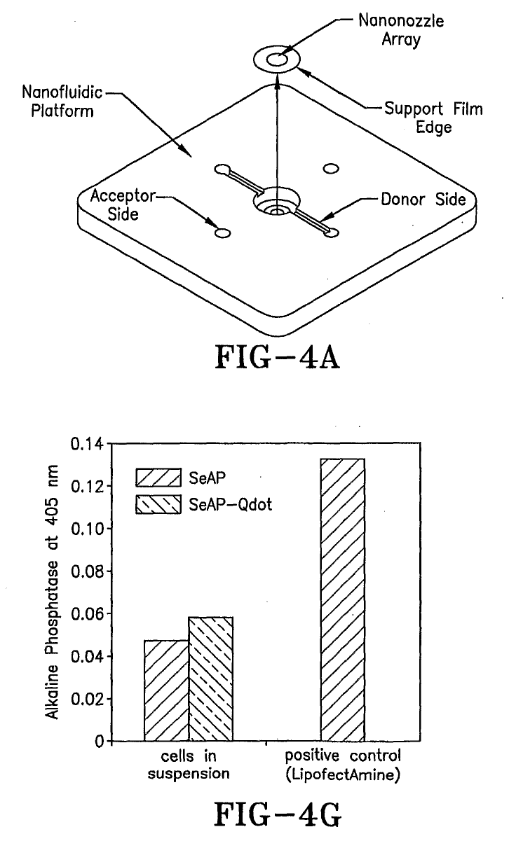

The EP gun cell patch can be used in two different ways to deliver drugs or genes into cells and/or tissues, as shown in FIGS. 3a and 3b. In FIG. 3a, cells are placed at a short distance away from the nanonozzle patch surface. At that location, the cells would experience a very low electric shock (Eo as in FIG. 2) during electrophoresis. A spacer can be used to control the spacing between the individual cells and the small end of the proximate nanonozzle. Momentum gained in the converging channel of the nanonozzle is used to insert particles conjugated with the drug or gene into the cells. Since the cells are not subject to any significant electric field strength, higher voltage and larger electrophoresis time can be applied.

In FIG. 3b, the cells are in contact with the outlet of the nanonozzle cell patch. Around the outlet area, cell membrane will experience very strong localized electrophoresis where an electric bias is applied. This, together with the electrophoretic mobility gained in the converging channels, would deliver the drug/genes into cells. Lower voltage and short duration (i.e., pulses) should be used in the strategy to avoid lysis of the cell.

For small genes or drugs, conjugated rigid particles (e.g., Quantum dots and gold nanoparticles) can be used as the carriers. While traveling inside the converging nanonozzles, these carriers are able to gain high enough momentum to overcome the cell membrane resistance, so drugs and/or genes can be delivered into cells. Optimizing the operation parameters, this method can be gentle enough that cell membrane is able to completely recover after a short period of delivery time. Large genes have long and flexible polymer chains. They often present in a supercoiled configuration with the radius of gyration in micrometers. The flexible chains can be stretched with external forces to form long and worm-like “nanowires” with the radius of gyration around 2 nm, which can pass through the intrinsic pores on cell membrane. The great velocity gradient inside the converging channel provides enough stress to stretch gene molecules from their equilibrium coiled conformation to the stretched conformation. The extent of stretching will depend upon both the flow stress and the relaxation time of the molecule conformation.

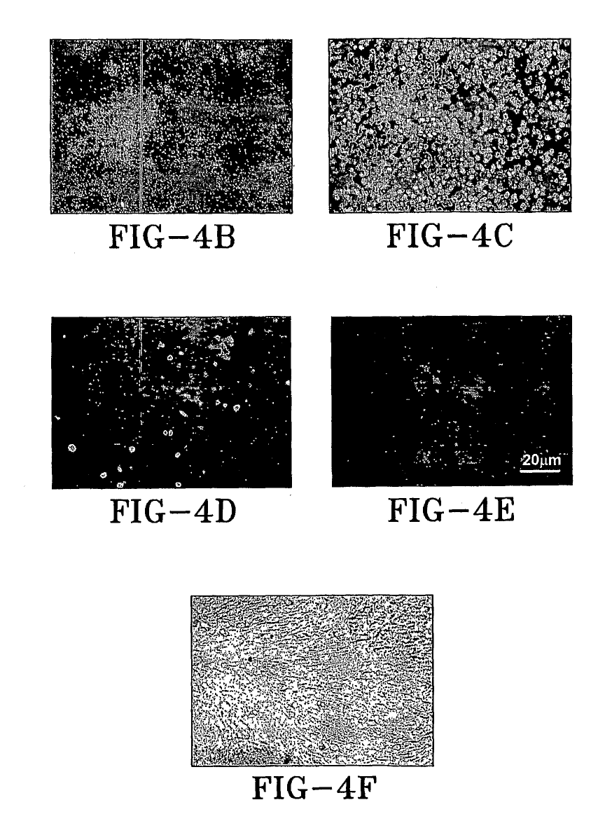

To demonstrate these strategies, both rigid colloid nanospheres of various sizes (i.e., SeAP conjugated QDots, and PS nanospheres with size of 50-200 nm, Polysciences, Inc) and flexible biomolecules (i.e., X-DNA, GFP and SeAP) were used. The nanonozzle array was placed in a miniaturized microfluidic platform as shown in FIG. 4a and sterilized under UV irradiation. The cells were immobilized using both gentle suction trapping and pre-culture on a track-etched membrane (PETE, GE Osmonics) with the pore size of 450 nm to 1 μm. The assembled nanonozzle cell patch was then placed in the reservoir located at the center, which was connected to both upstream and downstream channels. Gene solutions and medium were loaded to the connected upstream and downstream channels, respectively. After adding electric bias for a certain time period, cells with the track-etched membrane were removed and cultured in fresh medium. Samples were collected and gene expression was measured 48 hours after cell culture. All steps were carried out in a tissue culture hood under sterile conditions.

For large flexible molecules (i.e., %-DNA, 48.5 kbp, New England Biolabs), a dilute solution (−0.03 pg/ml, about 10⁻⁴ of the concentration at which the macromolecules completely fill the space without overlapping) prepared in Tris-EDTA buffer was used and labeled with a fluorescent dye (YOYO-1, Molecular Probes, Eugene, Oreg.) at a dye-base pair ratio of 1:5. Glucose (18%, w/w) and sucrose (40%, w/w) were added in the solution and the final viscosity of DNA solution was 30 cp so that the maximum relaxation time of %-DNA chain was about 1.9 second. DNA solution was loaded to the cathode side, while the anode side was loaded with buffer solution only. The nanonozzle exit was focused using an inverted epi-fluorescence microscope (TE 2000-S, Nikon) mounted underneath the microfluidic platform with a 100×/1.3 NA oil immersion objective lens. Due to the negative charge carried on its chain, DNA molecules migrated from cathode to anode through the nanonozzle cell patch and images are captured. A large number of DNA molecules were observed immediately on the permeate side after loading the DNA sample and adding electric bias (shown in FIG. 4b). This experiment confirmed the delivery of large DNA molecules through converging nanochannels. The migration rate of DNA molecules depends on the dimensions and geometry of nanochannels, the physical characterization of DNA, and the electric field strength.

FITC conjugated Dextran (2M Dalton, Molecular Probes) was also used as a model drug. Dextran was delivered to cells using the EP gun set-up described above. The cells were washed with PBS and stained with propidium iodide (PI) to label the cell nuclei in red fluoresce. Cells were examined under florescence microscope using the FIX and Rhodamine filters. By compounding two images together, this experiment verified that Dextran could be successfully delivered into a large number of cells, as shown in FIG. 4c.

For small genes, PEGFP (BD Biosciences Clontech, Mountain View, Calif.) and pGeneGrip SeAP (Gene Therapy System, San Diego, Calif.) were used as model genes and QDots as the model carrier. Our preliminary results demonstrated that both GFP and SeAP plasmids conjugated on QDots can be delivered to NIH 3T3 fibroblast cells seeded on membrane by suction (cells in suspension) and expressed 48 h after EP gun gene delivery, as shown in FIGS. 4d and 4e. Most of cells survived after the treatment, as shown by the phase contrast image in FIG. 4f. The efficiency of gene delivery is quantified by the amount of secreted alkaline phosphatase (SeAP) expressed, as shown in FIG. 4g. In this experiment, electrical parameters were set as follows: voltage 50-100 V, pulse width 5-500 ms, frequency 1-100 Hz and total duration 1-3 s. Under these conditions, SeAP conjugated with QDots gave rise to a higher alkaline phosphatase expression than that of SeAP alone (shown in FIG. 4g). This suggests that the high momentum built inside nanochannels does help the delivery. The efficiency of our technique is about one-third of that of the conventional lipofectamine method. This is promising when considering the fact that NIH 3T3 is one of the most suitable cell lines for lipofectamine and the operation conditions have not been fully optimized. Applicability of this method to the hard-to-transfect cells, such as mouse embryonic stem cells, the human mesenchymal stem cells, and natural killer cells, is under investigation.

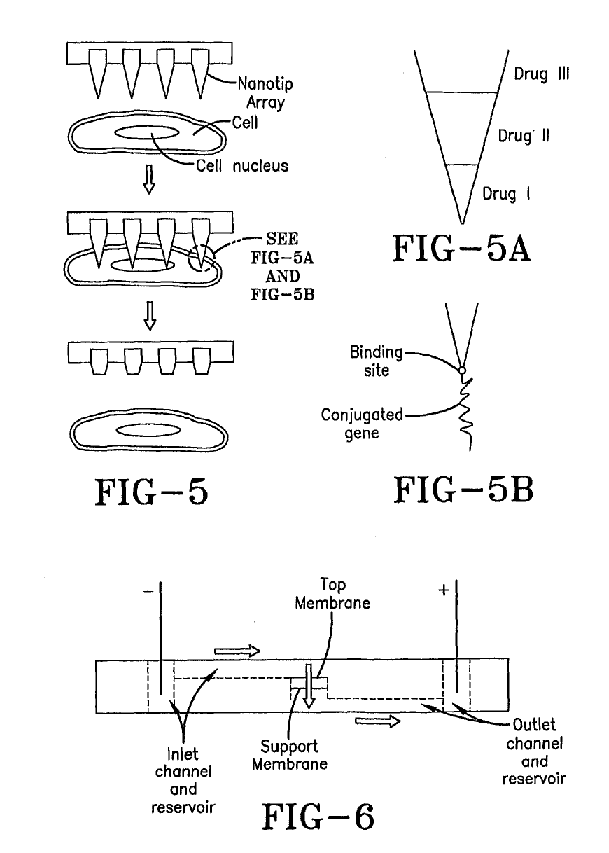

FIG. 5 also shows methods for using the polymer nanotips for drug/gene delivery. Nanotips are conjugated with drugs/genes. These conjugated nanotips penetrate into cells or even cell nuclei with gentle force. Different from other microinjection processes, this nanotip cell patch does not require a pullout step. The drug/gene is left inside the cell when the tip end of nanotips dissolves in the cell medium. The drug/gene is bound onto the nanotip by either directly conjugation on the surface of nanotips (FIG. 5b) or precipitating at the aperture of nanotips (FIG. 5a). The selection of binding strategies will vary with the materials to be delivered. In general, genes can be conjugated by covalent bond or van der Waal forces (e.g., avidin-biotin affinity binding) because of their simple and similar structures. Drugs will be fabricated as part of the aperture because of the complicated and widely various molecular structures for different drugs. Additionally, different drugs can be loaded by being embedded at different locations of nanotips, as shown in FIG. 5a. The geometry of nanotips and accurate manipulation prevent lethal damage to the host cells while the short and shallow penetration is enough for the delivery into cell or even its nuclei. Like nanonozzle cell patch, nanotip arrays also allow simultaneous treatment of a large population of cells. The dosage of drug/gene can be controlled by the total amount of samples and penetration depth of the nanotips. If necessary, multiple dosage or various delivery materials can also be injected into the targeted cells.

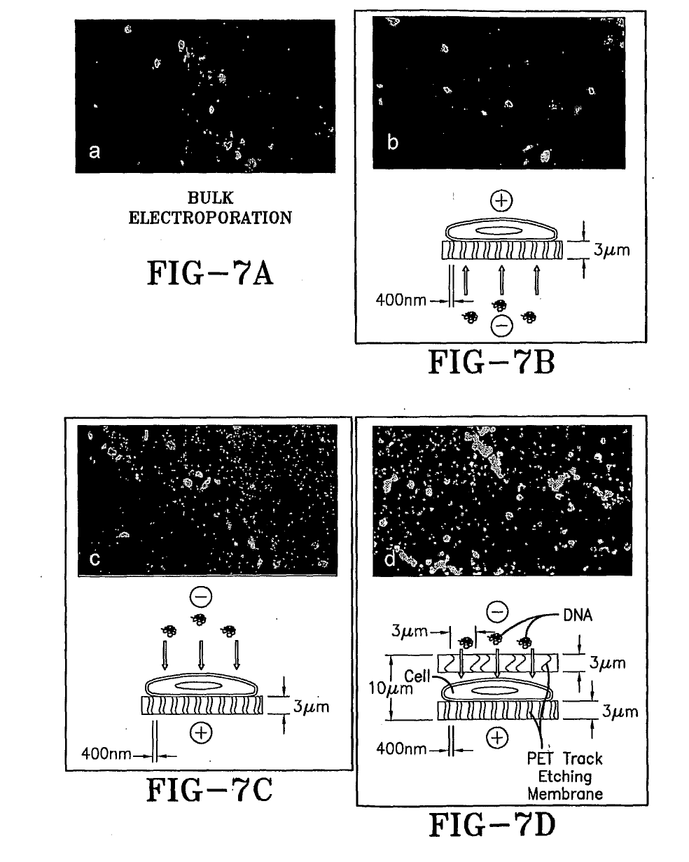

FIG. 6 illustrates a similar concept that has been successfully used for gene transfection to NIH 3T3 cells using a technique, referred to herein as “membrane sandwich electroporation” or “MSE”. First, a 3 mm diameter polyethylene terephthalate (PET) track etch membrane with the average pore size of 400 nm as the support membrane was placed in the middle of a 1 cm diameter reservoir located at the center of the microfluidic device. The reservoir is connected to both the inlet (top) and the outlet (bottom) channels, the channel size being 1 mm wide and 500 μm deep. A vacuum (10±1 in Hg) was used to trap the cells on the support membrane. Cell immobilization on a porous surface led to localized electroporation, allowing the use of low applied voltage to achieve dielectric breakdown of the cell membrane. Next, another 3 mm diameter PET track etch membrane with the average pore size of about 3 μm was placed on the top of the immobilized cells with a spacer of about 10 μm located between the two membranes. Then, Opti-MEM I reduced-serum medium was loaded in the channels and center reservoir, and the DNA sample was loaded in the inlet reservoir. After carrying out electroporation and a cell culture time of 24 to 48 hours, transfection efficiency was measured.

### Experimental Detail

### Plasmids and Cell Line

Plasmid pEGFP and NIH 3T3 fiberblasts were used as reporter gene and model cells. The plasmid pEGFP and PSEAP were prepared with an EndoFree Plasmid Maxi Kit from Qiagen (Valencia, Calif., USA) according to the manufacturer's instructions. NIH 3T3 cells (Mouse embryonic fibroblast cell line) were cultured in Dulbecco's modified Eagle's medium: Nutrient Mix F-12 (D-MEM/F-12) supplemented with L-glutamine (2 mM), sodium pyruvate (1 mM), and 10% (v/v) newborn calf serum (NCS). Cells were maintained in 25 cm² T-flasks at 37° C. with 5% CO₂ and subcultured using 0.25% (w/v) trypsin with EDTA 4Na. All cell culture reagents were purchased from Invitrogen (Carlsbad, Calif., USA).

### Electroporation Procedure

The electroporation conditions are given below in Table 1. In each experiment, the total cell number was about 1×10⁴, and the amount of DNA loaded was 0.5 μg. The pulse type was bipolar square wave. The NIH 3T3 cells were first plated on a 35 mm diameter plastic petri dish and allowed to grow for 2 days. Cells were then rinsed by trypsin-EDTA solution, washed with Dulbecco's Phosphate Buffered Saline (D-PBS) without calcium or magnesium, and adjusted to a final cell density of 1×10⁶ cells/ml in Opti-MEM I reduced-serum medium (w/o phenol red).

TABLE 1

| | Bulk electroporation | Localized cell |
|---|---|---|
| Method | (Single cuvette) [5] | electroporation and MSE |
| Amplitude (V/cm) | 1,600 | 35 |
| Pulse Frequency (Hz) | 40 | 1 |
| Pulse duration (ms) | 0.4 | 500 |
| No. of pulse | 1 | 5 |
| Electroporator | Bio-Rad Gene Pulser | Homemade microfluidic |
| | Xcell | cell |

A poly(ethylene terephthalate) (PET) track etch membrane with an average pore size of 400 nm was sealed in the microfluidic platform and used as a support membrane in the manner shown in FIG. 6. A 10 μl drop of cell suspensions (about 1×10⁴ cells) was loaded onto the support membrane, and cells were trapped on the support membrane using vacuum at the negative pressure of 10±1 in Hg. For the sandwich configuration, another PET track etch membrane with average pore size of about 3 μm was placed on the top of the immobilized cells with a spacer of about 10 μm between two membranes. Then, the inlet (top) and outlet (bottom) channels were filled with 100 μl of Opti-MEM I reduced-serum medium with and without DNA molecules, separately. 0.5 μg plasmid was used for each run of experiments. Finally, two thin silver wire electrodes were placed in inlet and outlet reservoirs and the two-step external electric-pulse program (as set for in Table 2) was applied to transfer DNA molecules into the cells. After 15 to 20 minutes, the support membrane with the cells was transferred to a 24-well plate and subsequently cultured in D-MEM/F-12 media with 10% NCS at 37° C./5% CO₂ until measuring the transfection efficiency (normally 24-48 hr).

TABLE 2

| | DNA Attraction [6] | Electroporation |
|---|---|---|
| Field strength (V/cm) | 3.5 | 35 |
| Pulse frequency (Hz) | 100 | 1 |
| Pulse duration (ms) | 5 | 500 |
| No. of pulse | 300 | 5 |

### Detection of Green Fluorescence Protein (GFP) Expression

The transfection efficiency of PEGFP was qualified by the percentage of the cells with green fluorescence. An inverted fluorescence microscopy (TS100, Nikon, USA) was used for detecting GFP expression and cell viability 24 hr after electroporation.

### Assay for Alkaline Phosphatase (AP) Activity

The transfection efficiency of PSEAP was quantified by the activity level of AP secreted by the transfected cells. Samples of culture media were collected 48 hours after electroporation and determined by a colorimetric assay based on the hydrolysis of p-nitrophenyl phosphate (pNPP). To do this, 100 μl of culture media and 30 μl of pNPP substrate solution (Sigma, USA) were added into each well of a 96-well plate. The plate was incubated in the dark for approximately 30 minutes at room temperature, and read at 405 nm on a multiwell plate reader (GENios Pro, Tecan, USA).

### Experimental Results

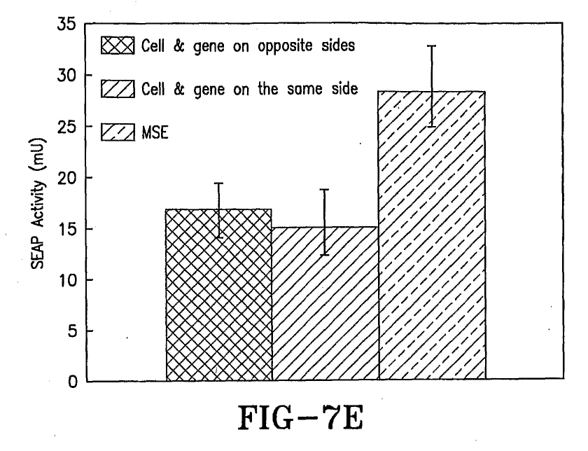

FIGS. 7a through 7e show various experimental set ups and results. As an initial baseline, FIG. 7a shows a photomicrograph illustrating the level of green fluorescence protein (GFP) expression provided by the conventional bulk electroporation method. In contrast to this, two different experiments were run using localized electroporation. In the first example, the cells and genes were placed on opposite sides of the support membrane, with photomicrograph results shown in FIG. 7b (and a schematic of the set up shown below the Fig.). The second example was run with the genes and the cells on the same side of the support membrane, with photomicrograph results shown in FIG. 7c (and the schematic set up shown below). Qualitative visual examination of the photomicrographs shows only slight improvement in the use of localized electroporation over bulk electroporation.

However, FIG. 7d shows an equivalent photomicrograph obtained when using the MSE setup method, the experimental set up again being illustrated below the photomicrograph. In this instance, most cells survived after the treatment, and GFP expression was much higher than in either bulk electroporation or in either of the localized cell electroporations.

Using another plasmid pSEAP, the levels of transgene expression mediated by localized cell electroporation and MSE were quantified. The results are presented as a bar graph in FIG. 7e, where bar 102 represents the secreted alkaline phosphatase (“SEAP”) expression when the “opposite side” localized electroporation was used, bar 104 represents the activity when “same side” localized electroporation was used, and bar 106 represents the SEAP activity after the MSE method. While the localized electroporation experiments show very similar results, the MSE technique had expression that was about 40% higher than localized cell electroporation.

During electroporation, cell permeabilization depends on the amplitude of electric pulses; while transportation of the polyanionic DNA molecules into the cells is driven by an electrophoretic force, and depends on the duration and number of electric pulses. The nanoscale pores in the support membranes in both the localized electroporation and MSE protocols allowed a focused electric field on the cell membrane, enhancing cell permeabilization at low electric voltage. However, negatively charged DNA molecules quickly migrate away from the negatively charged cell surface after the pulse duration because of electrically-repulsive forces. This limits gene transfer into the cells. However, in the MSE situation, the presence of a negatively charged PET track etch membrane on top of the cells, prevents the DNA molecules from moving away. Accordingly, the sandwiched membrane configuration provides better gene confinement near the cell surface and enhances gene transport into the cells.

Instead of using the high electric voltage and short pulse duration in bulk electroporation (as presented in Table 1), the localized electroporation and MSE experiments used five, bipolar, square-wave electric pulses, with very long duration of 500 ms at low field strength of 35 V/cm. This was observed to provide higher cell viability and better DNA transportation. While still being investigated, the applicability of the MSE method to primary cells and hard-to-transfect cells, such as mouse embryonic stem cells, and human blood mononuclear cells, would be expected to follow along the same sort of pathway.

Although the above examples all involve batch type cell patch drug/gene delivery devices, a flow-through set up was also designed, which could accomplish the same functions. An advantage of a flow-through electroporation system is that a large amount of cells (e.g., >10⁹ cells/ml) can be transfected simultaneously in short time (e.g., <10 seconds) to meet the quantity for future animal study or clinic trial.

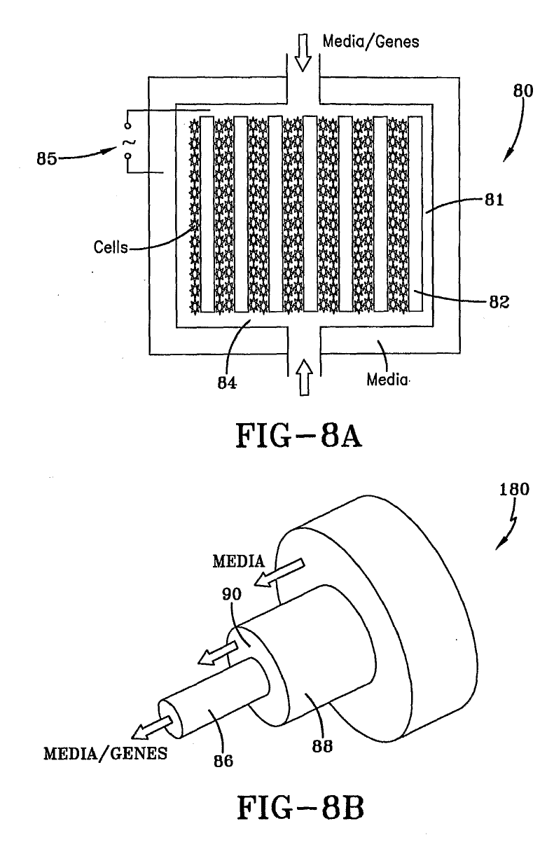

While the MSE system operates in a generally two-dimensional regime, the flow through configuration can be considered to be a three-dimensional membrane sandwich electroporation. Hollow-fiber bioreactors of a type generally known were used, although modified to allow for insertion of electrodes. In this way, the MSE protocol in such a flow-through hollow-fiber bioreactor can handle on the order of 10⁹ to 5×10¹⁰ cells in a single run. Referring now to FIG. 8a, a hollow-fiber bioreactor 80 typically has two compartments, the first compartment 82 defined by the internal volume of hollow fibers 81 and the second compartment 84 defined by a volume outside the hollow fibers 81. The cell and gene can be pre-mixed and flow inside the hollow fibers or fill separately inside and outside the hollow fibers, respectively. In both cases, media is supplied from the outside compartment. Electrodes 85 are inserted at the inlet or outlet of both flow streams. Since the hollow fibers 81 have porous walls, localized electroporation can be accomplished similar to the process of batch membrane sandwich electroporation.

As shown in FIG. 8b, a coaxial hollow-fiber bioreactor 180 of a generally known structure can be used. A coaxial hollow-fiber bioreactor 180 contains a fiber 86 within a fiber 88 to provide a third flow compartment 90, the third compartment defined by the annular volume internal to fiber 88 and external to fiber 86. The coaxial hollow-fiber bioreactor 180 is even closer to the setup for batch membrane sandwich setup. Cells would be caused to flow in the third compartment 90, while genes/drugs are supplied (in addition to media) in one of the other two compartments. In FIG. 8b, the compartment inside the smaller fiber 86 is used as this third compartment 90. Hollow fibers of this type have been known for years in industry in culturing cells, so the cells can be either immobilized or suspended in media for performing the MSE protocol.

Another type of flow-through electroporation, referred to herein as “hydrodynamic focusing electroporation”, is more general in nature. Because of this, other batch setups, such as nanonozzle array, can be integrated in the microfluidic platforms used for hydrodynamic focusing electroporation to further enhance gene delivery after electroporation.

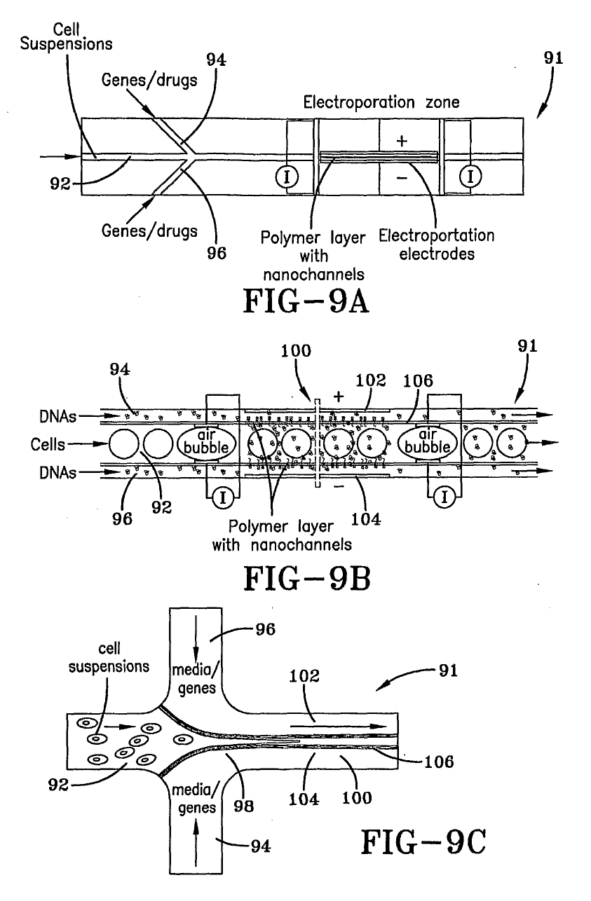

One embodiment of this system is shown schematically in FIGS. 9a-9c, which show the system from a top view (FIG. 9a) and in cross-section (FIG. 9b), as well as a cross-section view (FIG. 9c) of the flow patterns established. In this design, hydrodynamic focusing technique is applied to continuously supply both cells and drug/genes. The basic hydrodynamic focusing system 91 comprises three flow streams. A center flow stream 92 passes longitudinally through the reactor 91. A pair of side flow streams 94, 96 enter the center flow stream 92 obliquely and squeeze, or “hydrodynamically focus”, the center flow into a thin stream, the width of which may be controlled by adjusting the relative flow rates of the three flow streams 92, 94, 96. In some cases, this stream width can be controlled down to about 50 nm in the focusing zone 98. Cell suspensions are supplied in the center flow stream 92 and the drug or gene to be delivered can either be carried by either or both of the side flow streams 94, 96 or be pre-mixed with the cell suspensions in the center flow stream, in which case media would be used in the side flows 94, 96.

Directing attention to the cross-sectional view provided in FIG. 9b, an electroporation zone 100 is added downstream, shortly after the focusing zone 98. Electrical pulses are provided in the electroporation zone in a manner that would be known from operation of batch-type systems, the “reactants” (cells and drugs/genes) moving through the electroporation zone in an effectively plug-flow manner. Although the depiction provided shows the electrodes 102, 104 on the top and bottom channel surfaces, the electrodes could also be placed on opposing side surfaces. In many embodiments, the advantages already exhibited for the MSE protocol may be obtained by placing a pair of opposing polymer layers 106 containing nanochannels at or near the electroporation zone 100. In a particularly favored embodiment, the spacing between the polymer layers 106 will approximate the width of the focused center stream.

Besides the general advantage of flow-through electroporation (i.e., electroporation on a large population of cells), the hydrodynamic focusing flow-through electroporation has other benefits. By focusing the cells in the central stream 92, the opportunity for effective delivery of drugs/genes into cells in several ways: 1) cells can be forced in a line to pass the electroporation zone, ensuring the uniform electroporation on each cell; 2) the diffusion distance between the drug/gene and the cell is highly shortened to the micro/nanometer scale (in the focusing stream); and 3) the possible chock throat problem for cells in other focusing channels is minimized because of the application of moving boundary for the central flow stream.

What is claimed is:

1. A method for manufacturing a nanonozzle array, comprising the steps of:
providing an optical fiber bundle;
forming a nanotip array by removing material from the optical fiber bundle;
producing a replica mold of the nanotip array, using a poly(dimethylsiloxane);
casting a sacrificial nanotip template in the replica mold from a water-soluble material;
building up a composite sacrificial nanotip/nanonozzle array by spin coating a suitable material for the nanonozzle array onto the sacrificial nanotip template;
removing the nanonozzle array from the composite by dissolving the sacrificial nanotip template.

2. A method for delivering at least one drug or gene into a plurality of cells, comprising the steps of:
providing a nanonozzle array, wherein each nanonozzle in the array has a flow channel therethrough that converges from a first side of the nanonozzle array to a smaller end of the nanonozzle;
positioning the nanonozzle array proximate to the plurality of cells, the smaller ends facing the plurality of cells and a quantum of molecules of the at least one drug or gene in a fluid medium on the first side of the nanonozzle;
applying an electrical field of appropriate polarity from the first side of the nanonozzle array to a side of the plurality of cells opposite the nanonozzle array; and
using momentum gained by molecules in the converging flow channels to insert the molecules into one of the plurality of cells proximate the smaller ends.

3. The method of claim 2, wherein:
each of the molecules of the at least one drug or gene are conjugated to a rigid nanoparticle.

4. The method of claim 2, wherein:
the at least one drug or gene is a DNA molecule having a coiled conformation and a stretched conformation, and
the electrical field in the converging channel is sufficiently strong to transform the DNA molecules into the stretched conformation.

5. A method for delivering at least one drug or gene into a plurality of cells in a batch manner, comprising the steps of:
providing a first and a second nanoporous membrane, manufactured from a polymeric material and a microfluidic device;
immobilizing the plurality of cells on the first membrane in the microfluidic device;
providing a quantum of molecules of the at least one drug or gene in a fluid medium in the microfluidic device;
applying an electrical field of appropriate polarity across the membranes; and
using momentum gained by molecules in the nanopores of the membranes to insert the molecules into one of the plurality of cells proximate the smaller ends.

6. A method for delivering at least one drug or gene into a plurality of cells in a flow-through bioreactor, comprising the steps of:
providing the flow-through bioreactor having an inlet and an outlet and a flow chamber between the inlet and outlet, a nanoporous membrane positioned in the flow chamber;
flowing the plurality of cells from the inlet to the outlet;
providing a quantum of molecules of the at least one drug or gene in a fluid medium in the flow chamber;
applying an electrical field of appropriate polarity in the flow chamber; and
using momentum gained by molecules in the nanopores of the membranes to insert the molecules into the plurality of cells.

7. The method of claim 6, wherein:
the bioreactor is a hollow fiber bioreactor and the nanoporous membrane is provided by a plurality of hollow fibers.

8. The method of claim 7, wherein:
the hollow-fiber bioreactor is a coaxial hollow-fiber bioreactor having a pair of coaxial hollow fibers, and the plurality of cells flow from the inlet to the outlet in an annular volume between the outside of the smaller first hollow fiber and inside of the larger second hollow fiber.

9. The method of claim 6, wherein:
the bioreactor is a hydrodynamic focusing bioreactor with a central flow stream from the inlet to the outlet and a pair of side streams entering obliquely into the central flow stream, such that controlling the flow in the side streams focuses the central flow stream,
the cells flow in the central stream through an electroporation section where the electrical field is applied, and
the nanoporous membrane is positioned in the electroporation section.
